(19) United States
(12) Patent Application Publication
(10) Pub. No.: US 2026/0263697 A1
(43) Pub. Date: Sep. 10, 2026

(54) DELIVERY SYSTEM FOR EMERGENCY HYDROCORTISONE INJECTION

(71) Applicant: QOLIFY LLC, New York, NY (US)

(72) Inventors: Isabelle Brock, Chicago, IL (US); Roland Audette, North Kingstown, RI (US)

(21) Appl. No.: 19/168,906

(22) PCT Filed: Mar. 22, 2024
(86) PCT No.: PCT/US2024/021221

§ 371 Date: Sep. 25, 2025

Related U.S. Application Data
(60) Provisional application No. 63/454,769, filed on Mar. 27, 2023.

(51) Int. Cl. A61M 5/20; A61J 1/20; A61K 9/00; A61K 9/08; A61K 31/573

(52) U.S. Cl. CPC A61M 5/2066; A61J 1/2093; A61K 9/0019; A61K 9/08; A61K 31/573; A61M 2005/2026; A61M 2005/206; A61M 2205/502; A61M 2205/581

(57) ABSTRACT

An apparatus for automatic emergency hydrocortisone injection. The apparatus can include a pressing member configured to compress a vial to activate a solution in the vial. The apparatus can include a mixing member configured to shake the vial to mix the solution The apparatus can include an injection member configured to draw the solution from the vial and to inject the solution into an injection site of a person.

### CROSS-REFERENCE TO RELATED APPLICATIONS

This application claims the benefit of U.S. Provisional Application No. 63/454,769, filed Mar. 27, 2023, and titled “DELIVERY SYSTEM FOR EMERGENCY HYDROCORTISONE INJECTION” the disclosure of which is hereby incorporated herein by reference.

### TECHNICAL FIELD

The present disclosure relates generally to a medical device, and more particularly to a system configured to automatically mix and inject a dose of hydrocortisone.

### BACKGROUND

An adrenal crisis is a life-threatening medical emergency caused by insufficient levels of cortisol, which is a hormone produced and released by the adrenal gland. Patients prone to such episodes (e.g., people with Addison's) often carry an emergency injection kit-including a syringe, needle, and vial of hydrocortisone-in case they need to inject themselves with hydrocortisone to treat the adrenal crisis. Unfortunately, the process of manually injecting hydrocortisone is difficult.

The drug hydrocortisone is typically stored in a vial including two compartments to separate a powder form of hydrocortisone from a solution. Prior to injection, the patient manually crushes the vial to mix the hydrocortisone and solution. After swirling the vial to mix the powder and the solution, the patient wipes the vial with a prep pad for sterilization, opens the packaging of the syringe and needle, draws the solution into the syringe, and injects themselves with the needle to deliver the hydrocortisone.

This process is especially difficult to perform during an adrenal crisis. Typical symptoms include a severe drop in blood pressure, dizziness, lightheadedness, nausea, lethargy, confusion, and loss of consciousness. Moreover, failure to successfully complete the injection of hydrocortisone can be fatal. Accordingly, improved systems and techniques for delivering emergency hydrocortisone injections are desired.

### SUMMARY

Some aspects of the disclosure relate to a hand-held device that houses one or more vials of hydrocortisone along with mechanical and electrical components for automatically injecting the hydrocortisone into a person in case of an adrenal crisis. In embodiments, the process can be activated by the press of a button or in some cases by positioning the device against the skin of the person. During the administration process, the vial is crushed by a first syringe assembly to combine the internal components of the vial-a powdered form of hydrocortisone and a liquid. A piezoelectric device shakes the vial to stir its contents, creating an injectable solution of the hydrocortisone. The first syringe assembly then draws the mixed solution into its chamber.

A pump transfers the solution from the first syringe assembly to a second syringe assembly. The hand-held device may communicate (e.g., via display or speaker) its real-time progress and/or instructions for the user before next steps are performed. If the device detects that the user has properly pressed the housing to their skin and further detects that sufficient fluid has been pumped into the chamber of the second syringe assembly, it automatically injects the solution into the person via the second syringe assembly.

The hand-held device may include a window or clear front housing for the user to view the operation of the internal components. Additionally, the housing may include an override button that allows the user to direct the device to perform the injection in the event of sensor malfunction. Still further, the hand-held device may be equipped with a backup mechanical function that allows the user to manually perform the injection in the event of other types of component malfunction.

The hand-held device provides an advantage in that it helps a person experiencing the effects of an adrenal crisis to self-administer hydrocortisone in a reliable, safe, and efficient manner. Though various types of medical devices with automated needle injection may be known, the inventors are not aware of any device capable of automatic solution preparation prior to injection including crushing the vial to break the compartment divider of its contents and shaking the vial to mix the contents into an injectable solution.

Aspects of the present disclosure relate to an apparatus for automatic emergency hydrocortisone injection, including a pressing member configured to compress a vial to activate a solution in the vial, a mixing member configured to shake the vial to mix the solution, and an injection member configured to draw the solution from the vial, and to inject the solution into an injection site of a person.

One aspect of the present disclosure further includes a first syringe assembly including the pressing member, wherein the first syringe assembly is mechanically and fluidly coupled with the vial, a piezoelectric device to vibrate the vial to mix the solution, and a second syringe assembly including the injection member, wherein the second syringe assembly is fluidly coupled with the first syringe assembly.

One aspect of the present disclosure further includes a motor controller configured, in response to activation of a control member to initiate an automatic injection process, to direct a first actuator to drive the first syringe assembly to crush the vial, direct the piezoelectric device to vibrate the vial to mix the solution, direct a second actuator to draw the solution from the vial into the first syringe assembly, direct a pump to draw the solution from the first syringe assembly into the second syringe assembly, and direct a third actuator to drive the second syringe assembly, including a spring-loaded needle, into the injection site of the person. In one aspect, the pump is a peristaltic pump.

One aspect of the present disclosure further includes a contact sensor configured to detect contact between the apparatus and the injection site of the person, and a fluid sensor configured to detect a threshold amount of the solution in the second syringe assembly, wherein in response to detecting the contact and detecting the threshold amount of the solution, the motor controller is configured to trigger initiation of the third actuator to drive the second syringe assembly into the injection site.

One aspect of the present disclosure further includes an override element configured to trigger initiation of the third actuator if at least one of the contact and the threshold amount of the solution is not detected. One aspect of the present disclosure further includes a mechanical failsafe assembly configured to actuate the second syringe assembly into the injection site via manual user operation. One aspect of the present disclosure further includes a housing including a window for viewing an interior of the housing, a graphical user interface on the housing configured to display real-time visual instructions for the automatic emergency hydrocortisone injection, and a speaker configured to play real-time audio instructions for the automatic emergency hydrocortisone injection.

In one aspect, the vial is a hydrocortisone vial. In one aspect, the vial comprises two compartments including a first compartment with powdered hydrocortisone and a second compartment with a fluid to mix with the powdered hydrocortisone to create the solution.

A variety of additional inventive aspects will be set forth in the description that follows. The inventive aspects can relate to individual features and to combinations of features. It is to be understood that both the forgoing general description and the following detailed description are exemplary and explanatory only and are not restrictive of the broad inventive concepts upon which the embodiments disclosed herein are based.

### BRIEF DESCRIPTION OF THE DRAWINGS

The accompanying drawings, which are incorporated in and constitute a part of the description, illustrate several aspects of the present disclosure. A brief description of the drawings is as follows:

FIG. 1 is a perspective view depicting a vial of medicament, in accordance with the prior art.

FIG. 2 is a diagrammatic view depicting a user crushing the vial of FIG. 1 affect a mixing between a liquid and solid portion of the medicament.

FIG. 3 is a top view of the cap of the vial of FIG. 1 including a small tab.

FIG. 3A is a diagrammatic view depicting a user removing the small tab of FIG. 3.

FIG. 4 is a diagrammatic view depicting a user drawing the medicament from the vial of FIG. 1 into a syringe.

FIG. 5 is a diagrammatic view depicting a user injecting the medicament from the syringe of FIG. 4 into an injection site.

FIG. 6 is a plan view of an apparatus for medicament mixing and delivery, in accordance with an embodiment of the disclosure.

FIG. 7 is a perspective view of the apparatus of FIG. 6, in accordance with an embodiment of the disclosure.

FIG. 8 is a schematic view depicting an apparatus for automatic medicament mixing and delivery, in accordance with an embodiment of the disclosure.

FIG. 9 is a diagrammatic view depicting a user positioning the apparatus of FIG. 6 for delivery of medicament at an injection site, in accordance with an embodiment of the disclosure.

### DETAILED DESCRIPTION

Reference will now be made in detail to exemplary aspects of the present disclosure that are illustrated in the accompanying drawings. Wherever possible, the same reference numbers will be used throughout the drawings to refer to the same or like parts.

Adrenal glands, which are located above the kidneys, produce important hormones that are used by the body for some of its most basic functions. Specifically, the adrenal glands have two primary tasks. The first task is to produce adrenaline, a hormone which is typically created in times of stress. The second task is to produce two types of steroid hormones: cortisol and aldosterone.

Cortisol aids the body in dealing with stress, in particular it controls blood pressure and heart rate, controls how the immune system deals with viruses, bacteria and other threats, regulates a flow of sugar into the bloodstream, and adjusts how the body breaks down carbohydrates, proteins and fats. Aldosterone maintains a balance of sodium and potassium in the bloodstream, which aids in controlling blood pressure and the balance of fluids.

When the adrenal glands fail to produce enough of these hormones, some of these basic functions can break down. In particular, symptoms common to adrenal insufficiency can include fatigue, muscle weakness, low appetite, weight loss, and pain, among others. In some cases, the adrenal insufficiency (also referred to as adrenocortical insufficiency or hypocortisolism), can result in an acute, potentially life-threatening situation, sometimes referred to as an adrenal crisis.

In some cases, adrenal insufficiency can result when the adrenal glands are damaged, such that they are unable to produce the amount of cortisol and/or aldosterone needed by the body. This condition is typically referred to as Addison's disease. In other cases, adrenal insufficiency can be indirectly caused by abnormal production of adrenocorticotropin (ACTH) by the pituitary gland, which in turn signals the adrenal glands to produce cortisol when needed. In yet other cases, adrenal insufficiency can be indirectly caused by an abnormal production of a hormone called corticotropin-releasing hormone (CRH) in the hypothalamus, which in turn signals the pituitary gland to produce ACTH.

Although adrenal insufficiency is generally considered a rare condition, it is believed to be largely underdiagnosed, potentially resulting in life-threatening adrenal crises. Even with a proper diagnosis, adrenal insufficiency increases the mortality rate of a patient by a factor of four, largely because of the lack of availability of emergency kits, combined with the complexity of administering medication to address an adrenal crisis.

A typical emergency kit includes a syringe, needle and vial of hydrocortisone. Where an emergency kit is available, and the patient is familiar with the procedure for mixing and administering the hydrocortisone, self-treatment can be complicated by extreme muscle weakness and other neurological symptoms that typically accompany an adrenal crisis. If not treated promptly, the muscle weakness and neurological symptoms may progressively get worse over a span of between about 10 to about 30 minutes, after which the patient may slip into a coma and die. Unfortunately, the current procedure for the administration of a hydrocortisone solution typically takes about 15 minutes.

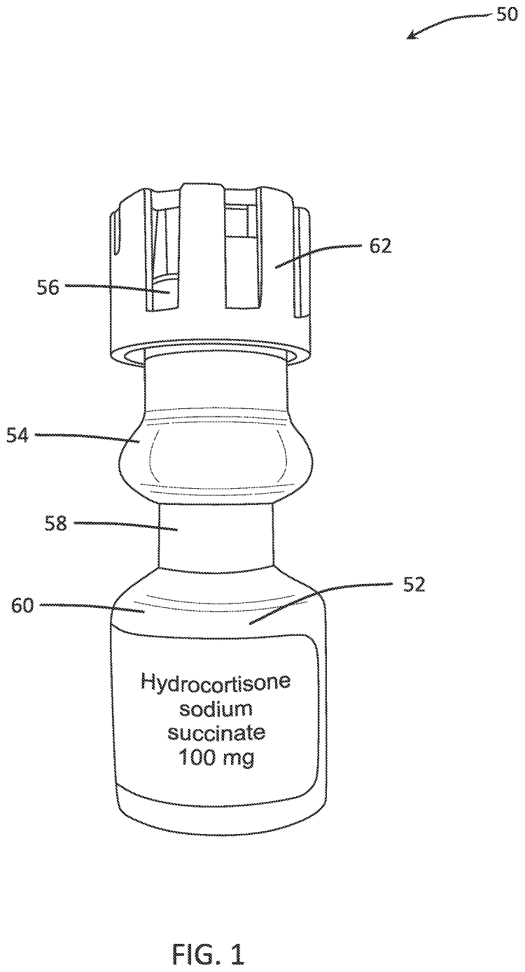

Referring to FIG. 1, a vial of hydrocortisone 50 is depicted. As depicted, the drug hydrocortisone (e.g., sometimes marketed under the name Solu-Cortef®), is typically stored in a vial 52 (e.g., sometimes referred to as an Act-O-Vial®). The vial 52 typically includes a first chamber 54 (e.g., containing a liquid) positioned between a first plunger 56 and a second plunger 58, and a second chamber 60 (e.g., containing the drug in a powdered form) isolated from the first chamber 54 by the second plunger 58. A cap 62 positioned on a top of the vial 52 can interface with the first plunger 56, such that pressing down on the cap 62 causes the first plunger 56 to be depressed into the first chamber 54. As the fluid in the first chamber 54 is generally incompressible, the fluid in the first chamber 54 will cause the second plunger 58 to be depressed into the second chamber 60, which contains some compressible gas along with some component of the drug in a solid form. Thereafter, the fluid from the first chamber 54 can flow into the second chamber 60 to be mixed with the solid component of the drug to form a liquid drug solution, which can then be injected into the patient.

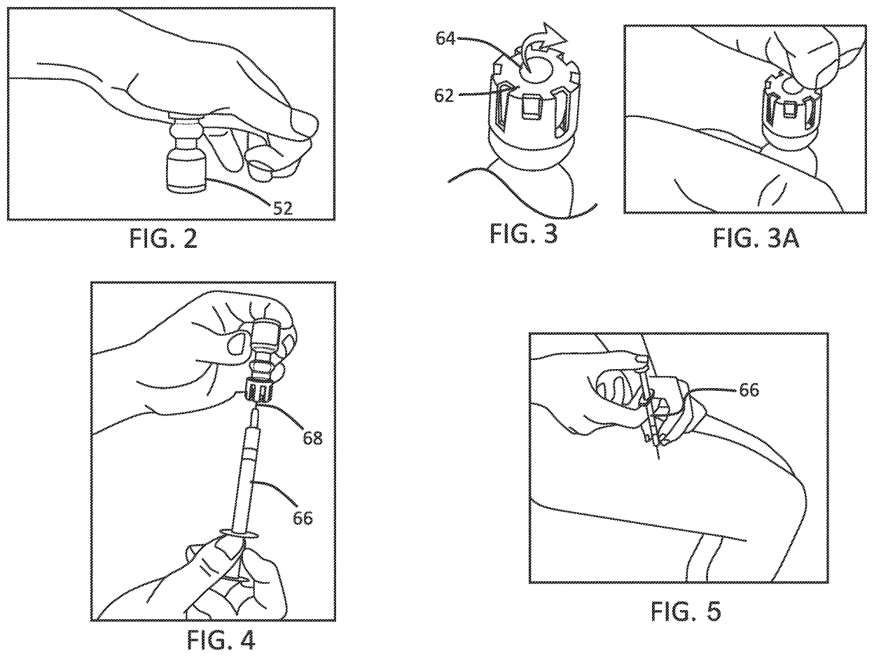

With additional reference to FIG. 2, in addition to washing hands and checking the expiration date of the medication, manual injection of hydrocortisone begins with pressing on the cap 62 of the vial 52 to form the drug solution. To ensure mixing, the vial 52 is then typically shaken for several seconds until the medication turns clear, which is an indication that the hydrocortisone is fully mixed. A cloudy mixture may be an indication that further agitation of the vial 52 may be required to ensure that the hydrocortisone is fully mixed.

With reference to FIGS. 3 and 3A, a small tab 64 located on the top of the cap 62 is removed, thereby exposing a top surface of the first plunger 56. Thereafter, the hydrocortisone solution can be withdrawn from the vial 52, for example via a syringe 66, as depicted in FIG. 4. Preferably the top surface of the first plunger 56 and the needle 68 of the syringe 66 are sterilized prior to insertion of a needle 68 through the first plunger 56.

After checking for and removal of any air bubbles, the hydrocortisone solution can be injected into the leg of a patient. Ideally the skin around the area of the injection is sterilized (e.g., via an alcohol wipe, or the like). In some cases, it may be beneficial to press down on the skin around the area of the injection prior to insertion of the needle. The needle is ideally inserted straight down into the thigh of the patient in a single quick motion, and the plunger 70 of the syringe 66 is depressed, thereby injecting the hydrocortisone solution into the muscle tissue of the leg. For most patients, a typical dosage includes the uniform injection of 2 mL of hydrocortisone solution (e.g., containing approximately 100 mg of hydrocortisone sodium succinate) over about 10 seconds; although for smaller patients (e.g., children, etc.) a dosage 1 mL administered over the same period of time may be preferable.

As hydrocortisone in a liquid form typically only has a shelf life of about 3 days, the administration of hydrocortisone in the event of an adrenal crisis necessarily involves the step of mixing the hydrocortisone prior to injection. Given that the patient may have as few as 10 minutes before losing consciousness, the overall time required to properly sterilize, mix and inject hydrocortisone is problematic. Further compounding the problem is the loss of dexterity and neurological function as the conditions of the adrenal crisis worsen. In many cases, it is simply not possible for a patient to self-administer hydrocortisone before slipping into a coma.

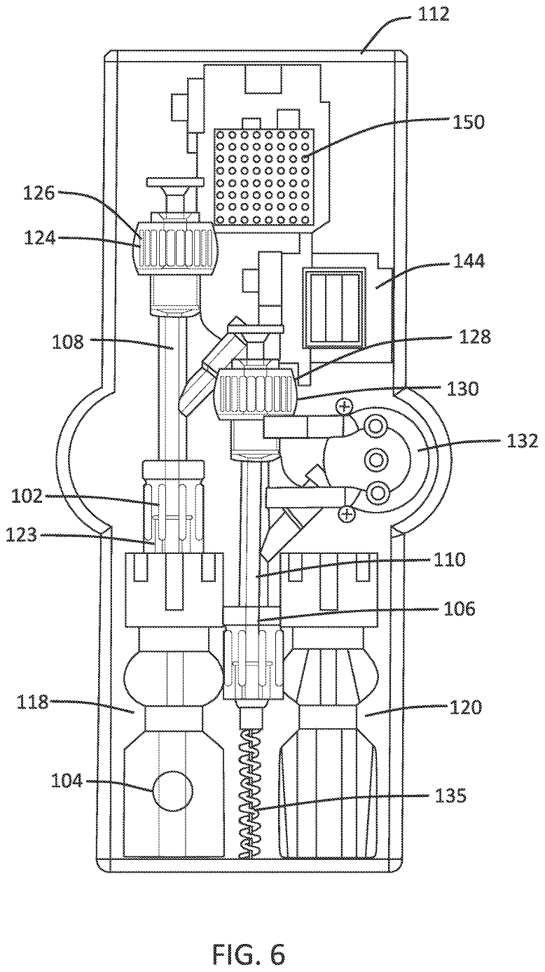

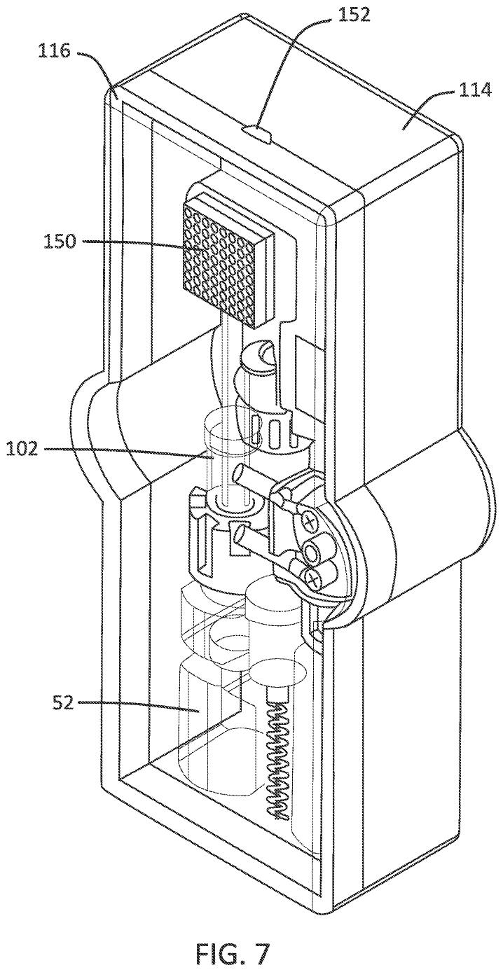

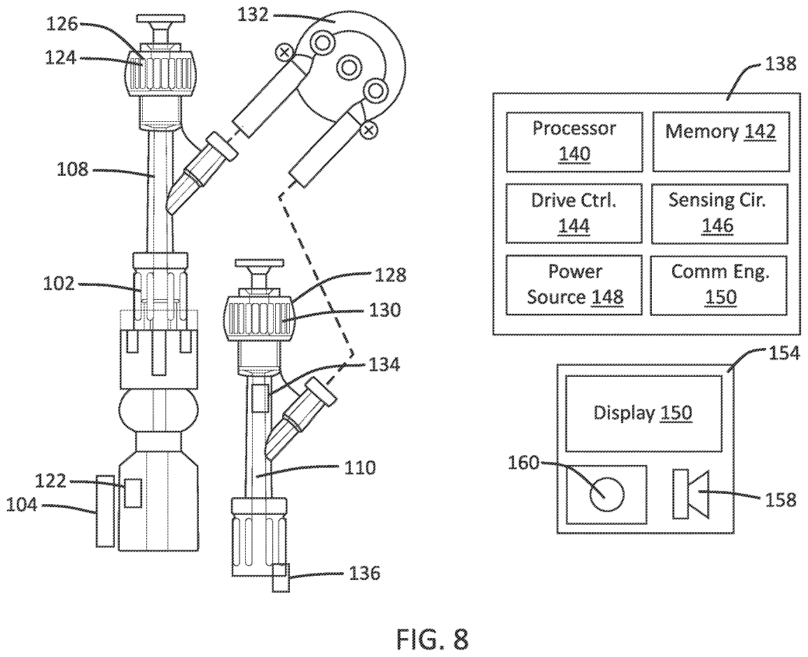

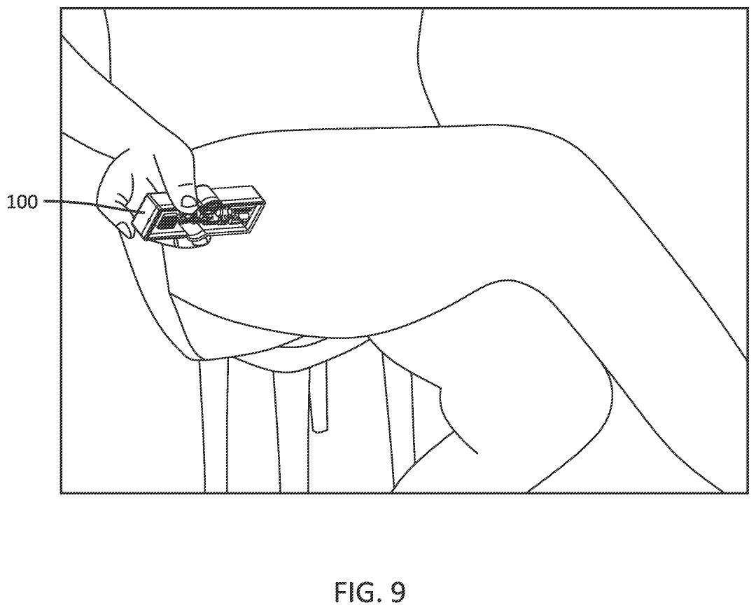

With additional reference to FIG. 6, Applicants of the present disclosure have invented an apparatus 100 configured to automate portions of the emergency hydrocortisone mixing and injection to address this problem, thereby enabling a patient to self-administer a lifesaving dose of hydrocortisone, for example, through the simple press of an activation button or by positioning the apparatus 100 on their leg at the injection site. Although the apparatus 100 is described as dispensing emergency hydrocortisone, the apparatus 100 is equally applicable to other types of medicaments, supplements and treatments normally contained in the type of compressible vial described above.

In embodiments, the apparatus 100 can include a housing 112 containing a vial 52, a pressing member 102 configured to compress the vial 52 to activate a solution in the vial 52, a mixing member 104 configured to shake the vial to mix the solution, and an injection assembly 106 configured to draw the solution from the vial 52 and inject the solution into an injection site of a person. In some embodiments, the injection assembly 106 can include a first syringe assembly 108 configured to draw solution from the vial, and a second syringe assembly 110 configured to inject the solution into an injection site of a person.

In some embodiments, the housing 112 can generally include a base portion 114 and a cover portion 116. In some embodiments, either or both of the base portion 114 or cover portion 116 can include a transparent or translucent window (e.g., the cover portion can be constructed of a clear material, etc.), enabling a user to see portions of the interior of the housing 112 (e.g., viewing operations of the mixing and injection, checking expiration dates, etc.). In some embodiments, the housing 112 can be substantially rectangular in shape, with opposing (e.g., arced, etc.) protrusions, which can be configured to provide an adequate gripping surface to properly position and hold the apparatus 100 against the skin of the patient at the injection site.

As depicted, the housing 112 can include a first vial receptacle 118, which can be configured to receive a compressible vial 52 of medicament, such as that described above. For example, in one embodiment, the first vial receptacle 118 can include one or more flexible fingers configured to temporarily flex under a natural material resiliency to receive and retain the vial 52. In some embodiments, the small tab 64 on the cap 62 of the vial 52 can be removed prior to positioning the vial 52 within the first vial receptacle 118. In some embodiments, the housing 112 can include a second vial receptacle 120 configured to receive a second vial of medicament, for additional dosing should a second vial of medicament be needed.

The pressing member 102 can be configured to apply a pressure or force to the cap 62 of the vial 52, in order to activate the first and second plungers 56, 58 to enable mixing of the medicament solution. In embodiments, the pressing member 102 can be an electric linear actuator, worm drive or the like configured to convert energy through the use of a driving mechanism into linear or rotary motion to apply a pressure or force to the top of the vial 52. In some embodiments, the pressing member 102 can be configured to apply a force of up to about 10 pounds, or between about 10 pounds and about 20 pounds, although other plunger force outputs are also contemplated.

With the cap 62 of the vial 52 depressed by the pressing member 102, the liquid portion of the medicament contained in the first chamber 54 can flow into the second chamber 60 containing the solid portion of the medicament, thereby enabling mixing between the liquid and solid components. In some embodiments, to encourage a proper mixing of the components, the apparatus 100 can include a mixing member 104. In some embodiments, the mixing member can be a piezoelectric vibrator, or other type of actuation that uses a piezoelectric effect to generate mechanical motion. In some embodiments, the mixing member 104 comprises a piezoelectric material, such as a quartz, which generates mechanical motion when subjected to an electric field. Other types of mixing members 104 are also contemplated.

With some types of medicaments, proper mixing of the liquid and solid components is indicated by the resulting solution having a particular attribute (e.g., turning clear, etc.). In some embodiments, the apparatus 100 can include a medicament sensor 122 (e.g., optical sensor, etc.) configured to gather information regarding a particular attribute of the medicament. For example, in one embodiment, the apparatus 100 can determine if the medicament has turned clear to determine if the mixing process is complete, and the process for the administration of the medicament may progress to the next step.

With mixing complete, the medicament may be withdrawn from the vial 52 into the first syringe assembly 108. In embodiments, the first syringe assembly 108 can include a needle, barrel and plunger. The barrel can be a cylindrical container configured to hold the fluid medicament. The needle can include a sharp, pointed tip attached to the barrel for inserting into the vial 52. The plunger can be a piston-like component that fits snugly inside the barrel and controls the flow of medicament. In embodiments, the first syringe assembly 108 can include two actuation assemblies: a first actuation assembly 124 configured to urge the needle through the first plunger 56 of the vial 52, thereby enabling a portion of the first syringe assembly 108 to be inserted into the vial 52, and a second actuation assembly 126 configured to move the plunger relative to the barrel, thereby enabling the medicament to be withdrawn from the vial 52 into the barrel of the first syringe assembly 108. In some embodiments, the first and second actuation assemblies 124, 126 can be powered by a common electric motor.

In some embodiments, a sterilization assembly 123, for example positioned in close proximity to the cap 62 of the vial 52 can be configured to sterilize the top of the first plunger 56 and the needle of the first syringe assembly 108 prior to insertion of the needle into the vial 52. For example, in one embodiment, the sterilization assembly can be configured to apply an isopropyl alcohol solution to portions of the vial 52 and first syringe assembly 108.

Thereafter, the medicament can be transferred to the second syringe assembly 110. In some embodiments, a transfer of medicament from the first syringe assembly 108 to the second syringe assembly 110 can be performed by a pump 132. In one embodiment, the pump 132 can be a peristaltic type pump, or a positive displacement pump that uses a rotating roller or shoe to compress and move a flexible tube, creating suction at one end and discharge at the other, although other types of pumps configured to isolate the fluid from the pumping mechanism to inhibit contamination are also contemplated. In other embodiments, transfer of the medicament can be performed by the first and second syringe assemblies themselves, for example by a coordinated movement of the plungers while a fluid path is provided between the first syringe assembly 108 and the second syringe assembly 110. In some embodiments, one or more air permeable wicks may be provided on the injection assembly 106 to enable air bubbles trapped in the medicament solution to escape prior to injection.

Like the first syringe assembly, the second syringe assembly 110 can include a needle, barrel, plunger, and first and second actuation assemblies. In embodiments, the first actuation assembly 128 can be configured to urge the needle through the skin of the patient into the injection site. In some embodiments, the apparatus 100 can include a second sterilization assembly configured to provide sterilization for the needle of the second syringe assembly 110 and/or the injection site, prior to injection. The second actuation assembly 130 can be configured to move the plunger relative to the barrel, thereby enabling the medicament contained within the barrel to be dispensed through the needle and into the tissue of the patient. As an aid in timing of the injection, in some embodiments, the apparatus 100 can include a fluid sensor 134 configured to determine when a threshold quantity or amount of the medicament is present within the barrel of the second syringe assembly 110. After injection, in embodiments, the second syringe assembly 110 can be configured to withdraw the needle. For example, in some embodiments, the needle can be biased by a spring 135 to the retracted position.

To aid users in proper positioning of the apparatus 100 during injection, in some embodiments, the apparatus can include a contact sensor 136, or other form of electronic device configured to detect skin conductance, or the electrical resistance of the skin. In some embodiments, the contact sensor 136 can be used to ensure that the base portion 114 is properly positioned with respect to the skin of a patient prior to actuation of the first and second actuation assemblies 128, 130 of the second syringe assembly 110.

In embodiments, the various electrical components driving aspects of the medicament delivery can be controlled by a controller 138. The controller 138, alternatively referred to as an electronic control unit (ECU), is configured to perform arithmetic and logical operations, and is responsible for executing instructions from software programs and controlling other components. As used herein, the term controller can comprise or include various modules or engines, each of which is constructed, programmed, configured, or otherwise adapted to autonomously carry out a function or set of functions. The term “engine” as used herein is defined as a real-world device, component, or arrangement of components implemented using hardware or as a combination of hardware and software, such as by a microprocessor system and a set of program instructions that adapt the engine to implement a particular functionality, which (while being executed) transform the microprocessor system into a special-purpose device.

An engine can also be implemented as a combination of hardware and software, with certain functions facilitated by hardware alone, and other functions facilitated by a combination of hardware and software. Accordingly, each engine can be realized in a variety of physically realizable configurations, and should generally not be limited to any particular implementation exemplified herein, unless such limitations are expressly called out. In addition, an engine can itself be composed of more than one sub-engine, each of which can be regarded as an engine in its own right. Moreover, in the embodiments described herein, each of the various engines corresponds to a defined autonomous functionality; however, it should be understood that in other contemplated embodiments, each functionality can be distributed to more than one engine. Likewise, in other contemplated embodiments, multiple defined functionalities may be implemented by a single engine that performs those multiple functions, possibly alongside other functions, or distributed differently among a set of engines than specifically illustrated in the examples herein.

In some embodiments, the controller 138 can include a processor 140, memory 142, drive controller 144, sensing circuitry 146, power source 148, and communications engine 150. In embodiments, the processor 140 can include fixed function circuitry and/or programmable processing circuitry. The processor 140 can include any one or more of a microprocessor, a controller, a DSP, an ASIC, an FPGA, or equivalent discrete or analog logic circuitry. In some examples, the processor 140 can include multiple components, such as any combination of one or more microprocessors, one or more controllers, one or more DSPs, one or more ASICs, or one or more FPGAS, as well as other discrete or integrated logic circuitry. The functions attributed to the processor 140 herein may be embodied as software, firmware, hardware or any combination thereof.

The memory 142 can include computer-readable instructions that, when executed by the processor 140 cause the controller 138 to perform various functions. The memory 142 can include volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

The drive controller 144 can include instructions to control the electrical components of the apparatus 100. For example, in some embodiments, the drive controller 144 is configured to send and possibly receive electrical signals to the pressing member 102, mixing member 104, first and second actuation assemblies 124, 126, pump 132, and first and second actuation assemblies 128, 130 according to a timing or sequence of the medicament administration process. In some embodiments, the timing of the activations of the various components can be driven at least partially by the receipt of one or more signals from the sensing circuitry 146.

In embodiments, the sensing circuitry 146 can be configured to sense one or more signals related to the medicament and/or positioning of the apparatus 100 relative to an injection site. For example, the sensing circuitry 146 can include or can be operable with one or more sensors, including the medicament sensor 122, fluid sensor 134, and contact sensor 136.

The power source 148 is configured to deliver operating power to the components of the controller 138. The power source 148 can include a battery and a power generation circuit to produce the operating power. In some examples, the battery is rechargeable to allow extended operation. The power source 148 can include any one or more of a plurality of different battery types, such as nickel cadmium batteries and lithium ion batteries.

The communications engine 150 can include any suitable hardware, firmware, software, or any combination thereof for communicating with other components and/or external devices. Through either a wired (e.g., via port 152) or wireless connection (e.g., Bluetooth, etc.), the communication engine 150 facilitate communication with a network communicating device or computer network. For example, in one embodiment, the communications engine 150 can be configured to receive software updates, as well as to transfer information stored in the memory 142, including an event log which may detail actions taken by the apparatus or information gathered by the sensors, including a date and time stamp. In embodiments, port 152 can also serve as a receptacle for recharge of the power source 148.

In some embodiments, the controller 138 can be in communication with a user interface 154, which can be configured to enable user interaction with the controller 138. For example, in one embodiment, the user interface 154 can include a display 156 (e.g., LED display, etc.), an audio output device 158, and a button 160 (e.g., keypad, soft start activation button, etc.). In some embodiments, a user can press the button 160 to initiate the medicament mixing and administration procedure. In some embodiments, the at least one button 160 can serve as an override element, enabling delivery of the medicament, where any of the sensors (e.g., medicament sensor, 122 fluid sensor 134, or contact sensor 136) indicate that the next step in the procedure or positioning of the apparatus have not met a defined threshold. Accordingly, in some embodiments, advancements through the various medicament administration steps of crushing the vial, mixing the medicament, transfer of the medicament through the injection assembly, and injection of the medicament into the patient can be advanced manually through the override element.

In other embodiments, the physical steps taken by the apparatus 100 can be automated after the button 160 or contact sensor 136 triggers and initiation of the medicament administration process. In some embodiments, at least one of the display 156 and/or audio output device 158 can provide instructions to the user in order to guide the user through any steps necessary to ensure proper administration of the medicament (e.g., positioning of the apparatus 100 relative to the injection site, etc.). Further, in some embodiments, the user interface 154 can be configured to notify the user of the current step being taken in the medicament administration process (e.g., drug mixing, prepare for injection, countdown to injection, count down during injection, withdrawing needle, process complete, etc.).

In the event of a malfunction of any of the electrical actuation components, in some embodiments, the apparatus 100 can be provided with a mechanical failsafe to ensure that the medicament can still be administered. For example, in some embodiments, a user can remove the cover portion 116 to manually crush the vial 52, mix the medicament solution, advance the needle of the first syringe assembly 108 into the vial 52, draw the medicament solution into the first syringe assembly 108, transfer the medicaments solution to the second syringe assembly 110, advance the needle of the second syringe assembly 110 into the injection site, and advance the plunger of the second syringe assembly 110 to affect injection of the medicament into the injection site. In other embodiments, portions of the apparatus 100 can be disassembled from the housing 112 for manual use.

Having described the preferred aspects and implementations of the present disclosure, modifications and equivalents of the disclosed concepts may readily occur to one skilled in the art. However, it is intended that such modifications and equivalents be included within the scope of the claims which are appended hereto.

1. An apparatus for automatic emergency hydrocortisone injection, comprising:
a pressing member configured to compress a vial to activate a solution in the vial;
a mixing member configured to shake the vial to mix the solution; and
an injection member configured to draw the solution from the vial, and to inject the solution into an injection site of a person.

2. The apparatus of claim 1, further comprising:
a first syringe assembly including the pressing member, wherein the first syringe assembly is mechanically and fluidly coupled with the vial;
a piezoelectric device to vibrate the vial to mix the solution; and
a second syringe assembly including the injection member, wherein the second syringe assembly is fluidly coupled with the first syringe assembly.

3. The apparatus of claim 2, further comprising:
a motor controller configured, in response to activation of a control member to initiate an automatic injection process, to:
direct a first actuator to drive the first syringe assembly to crush the vial;
direct the piezoelectric device to vibrate the vial to mix the solution;
direct a second actuator to draw the solution from the vial into the first syringe assembly;
direct a pump to draw the solution from the first syringe assembly into the second syringe assembly; and
direct a third actuator to drive the second syringe assembly, including a spring-loaded needle, into the injection site of the person.

4. The apparatus of claim 3, further comprising:
a contact sensor configured to detect contact between the apparatus and the injection site of the person; and
a fluid sensor configured to detect a threshold amount of the solution in the second syringe assembly;
wherein in response to detecting the contact and detecting the threshold amount of the solution, the motor controller is configured to trigger initiation of the third actuator to drive the second syringe assembly into the injection site.

5. The apparatus of claim 4, further comprising:
an override element configured to trigger initiation of the third actuator if at least one of the contact and the threshold amount of the solution is not detected.

6. The apparatus of claim 4, further comprising:
a mechanical failsafe assembly configured to actuate the second syringe assembly into the injection site via manual user operation.

7. The apparatus of claim 3, wherein:
the pump is a peristaltic pump.

8. The apparatus of claim 1, further comprising:
a housing including a window for viewing an interior of the housing;
a graphical user interface on the housing configured to display real-time visual instructions for the automatic emergency hydrocortisone injection; and
a speaker configured to play real-time audio instructions for the automatic emergency hydrocortisone injection.

9. The apparatus of claim 1, wherein:
the vial is a hydrocortisone vial.

10. The apparatus of claim 1, wherein:
the vial comprises two compartments including a first compartment with powdered hydrocortisone and a second compartment with a fluid to mix with the powdered hydrocortisone to create the solution.
